(19) United States
(12) Patent Application Publication
(10) Pub. No.: US 2008/0255359 A1
(43) Pub. Date: Oct. 16, 2008

(54) Novel Chemical Process For the Synthesis of Quinoline Compounds

(76) Inventor: Charles Edward Wade, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/067,537

(22) PCT Filed: Sep. 26, 2006
(86) PCT No.: PCT/EP2006/009460

§ 371 Date: Mar. 20, 2008

(30) Foreign Application Priority Data
Sep. 28, 2005 (GB) 0519758.7

(51) Int. Cl. C07D 401/02; C07D 215/38; C07D 215/36

(52) U.S. Cl. 544/363; 546/153

(57) ABSTRACT

Disclosed is a novel, simplified and economic process for making 3-phenylsulphonyl quinolines with an amine group at position 8 of the quinoline ring system, including 3-phenylsulfonyl-8-piperazin-1-yl-quinoline in particular, in the absence of a palladium catalyst. Also disclosed is the crystallization of polymorphic forms of 3-phenylsulfonyl-8-piperazin-1-yl-quinoline.

This invention relates to a novel chemical process for the synthesis of quinoline compounds, in particular 3-phenylsulfonyl-8-piperazin-1-yl-quinoline and to the preparation of polymorphic forms thereof.

### BACKGROUND

WO 03/080580 (Glaxo Group Limited) describes the preparation of sulphonyl quinoline compounds including 3-phenylsulfonyl-8-piperazin-1-yl-quinoline (Example 16) in addition to two polymorphic forms of 3-phenylsulfonyl-8-piperazin-1-yl-quinoline (Form I; Example 51 and Form II; Example 52). These sulphonyl quinolines are disclosed as having affinity for the 5-HT₆ receptor and are claimed to be useful in the treatment of CNS and other disorders. 3-Phenylsulfonyl-8-piperazin-1-yl-quinoline is currently undergoing trials as a possible treatment for Alzheimer's disease.

WO 05/040124 (Glaxo Group Limited) describes a further polymorphic form of 3-phenylsulfonyl-8-piperazin-1-yl-quinoline characterised in that it possesses a higher melting point than Forms I and II. This further polymorphic form of 3-phenylsulfonyl-8-piperazin-1-yl-quinoline is referred to as Form III.

The process used for making 3-phenylsulfonyl-8-piperazin-1-yl-quinoline described in the prior art involves reacting 8-iodo-3-phenylsulphonyl quinoline and piperazine in the presence of a palladium catalyst. However, palladium is a precious metal and, therefore, its use in a process for making 3-phenylsulfonyl-8-piperazin-1-yl-quinoline results in that process being expensive to perform.

Furthermore, as palladium is toxic, precautions have to be taken when using the metal as a catalyst in chemical reactions and when disposing of the catalyst when the reaction is complete. Again, the implementation of such precautions makes a chemical process which uses palladium as a catalyst expensive to perform.

There is therefore a need for a concise and economical process for making 3-phenylsulphonyl quinolines with an amine group at position 8 of the quinoline ring system which avoids the use of a palladium catalyst.

### SUMMARY OF THE INVENTION

A novel, simplified and economic process has now been found for making 3-phenylsulphonyl quinolines with an amine group at position 8 of the quinoline ring system, including 3-phenylsulfonyl-8-piperazin-1-yl-quinoline, in the absence of a palladium catalyst. 3-Phenylsulfonyl-8-piperazin-1-yl-quinoline so made may then be optionally crystallised into one of its polymorphic forms.

In a first aspect of the invention there is therefore provided a process for the production of a compound of formula (I), or a salt thereof:

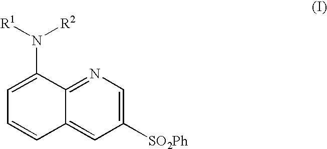

which comprises reacting a compound of formula (II):

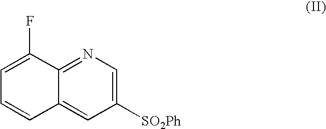

with a compound of formula R¹R²NH, in the presence of a base and a solvent; wherein:
R¹ and R² independently represent hydrogen or C₁₋₆ alkyl, or R¹ and R² together with the nitrogen atom to which they are attached form an optionally substituted 4 to 7 membered monocyclic heterocyclyl group which can optionally contain 1 or 2 further heteroatoms selected from O, N and S; and
Ph represents an optionally substituted phenyl group.

When R¹ and R² together with the nitrogen atom to which they are attached form an optionally substituted 4 to 7 membered monocyclic heterocyclyl group, the heterocyclyl group may be substituted by one or more (for example 1, 2 or 3) substituents, which may be the same or different, selected from the group consisting of halogen, C₁₋₆ alkyl, C₁₋₆ alkoxy, and —COC₁₋₆ alkyl.

When Ph is optionally substituted, the phenyl ring may be substituted by one or more (for example 1, 2 or 3) substituents, which may be the same or different, selected from the group consisting of hydroxyl, cyano, nitro, amino, amido, trifluoromethyl, trifluoromethoxy and C₁₋₆ alkyl.

The term “heterocyclyl”, unless stated otherwise, is intended to mean a 4 to 7 membered monocyclic saturated or partially unsaturated aliphatic ring containing 1 to 3 hetroatoms selected from oxygen, nitrogen or sulphur. Suitable examples of such monocyclic rings include azetidinyl, pyrrolidinyl, piperidinyl, oxypiperidinyl, piperazinyl, morpholinyl, thiomorpholinyl, diazepanyl, azepanyl, dihydroimidazolyl, tetrahydropyranyl, tetrahydrothiapyranyl and tetrahydrofuranyl. The term “base” is intended to mean any substance that can act as a proton acceptor. Potassium carbonate is an example of a base which is suitable for use in the process described above.

The term “solvent” is intended to mean any substance capable of dissolving another substance. N-propanol is an example of a solvent which is suitable for dissolving the reactants in the process described above.

In a particular embodiment of the process described in the first aspect of the present invention the process is performed in the absence of palladium and, more particularly, in the absence of any metal catalyst.

In certain embodiments of the first aspect of the invention herein described, R¹R²NH represents piperazine, and more particularly an excess of piperazine so that the Molar equivalence of piperazine to compound of formula (II) is greater than about 3 and in one embodiment is greater than about 5.

In one embodiment of the first aspect of the invention, the phenyl is unsubstituted.

In one embodiment of the first aspect of the invention, the compound of formula (II) is 8-fluoro-3-phenylsulfonylquinoline and the compound of formula R¹R²NH is piperazine.

In one embodiment of the first aspect of the invention, the reaction between a compound of formula (II) and R¹R²NH is carried out at a temperature between about 95 and about 105° C. In a further embodiment, the reaction is carried out at a temperature of about 100° C.

In one embodiment of the first aspect of the invention, the reaction is carried out under nitrogen.

In a further embodiment of the first aspect of the invention, there is provided a process for the preparation of 3-phenylsulfonyl-8-piperazin-1-yl-quinoline, which comprises reacting 8-fluoro-3-phenylsulfonylquinoline with an excess of piperazine in the presence of potassium carbonate and n-propanol at a temperature between about 95 and about 105° C.

A second aspect of the invention provides a process for the preparation of a compound of formula (II), or a salt thereof, which comprises reacting a compound of formula (III):

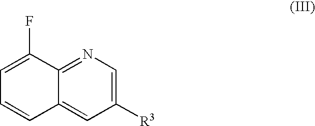

wherein R³ represents iodine or bromine;
with HSO₂Ph, or a salt thereof, in the presence of a diamine ligand, a metal catalyst, a base and a polar aprotic solvent.

The term “diamine ligand” is intended to mean any molecule containing two amino groups that can share electrons with a metal atom within a metal catalyst and thereby form a stable complex with the metal catalyst. Examples of diamine ligands include ethylenediamine-tetraacetate (EDTA) and N,N′-dimethylethylenediamine. The term “metal catalyst” is intended to mean any catalyst which contains at least one metal atom, for example copper iodide (Cu I).

The term “polar aprotic solvent” is intended to mean any hydrophilic solvent which has no hydrogen atoms that can be donated into a H-bond. Examples of polar aprotic solvents include dimethylsulfoxide, dimethylformamide and hexamethylphosphorotriamide.

The term “base” is intended to mean any substance that can act as a proton acceptor. Diisopropylethylamine is an example of a base which is suitable for use in the process of the second aspect of the invention described above.

In one embodiment of the second aspect of the invention, the compound of formula (III) is 8-fluoro-3-iodoquinoline.

In one embodiment of the second aspect of the invention, the salt of HSO₂Ph is benzenesulfinic acid sodium salt.

In one embodiment of the second aspect of the invention, the diamine ligand is N,N′-dimethylethylenediamine.

In one embodiment of the second aspect of the invention, the metal catalyst is CuI.

In one embodiment of the second aspect of the invention, the base is selected from the group consisting of diisopropylethylamine and potassium carbonate.

In one embodiment of the second aspect of the invention, the polar aprotic solvent is dimethylsulfoxide.

In one embodiment of the second aspect of the invention, the reaction is carried out at a temperature between about 60 and about 110° C. In a further embodiment, the reaction is carried out at a temperature between about 90 and about 105° C. In yet a further embodiment, the reaction is carried out at a temperature between about 100 and about 103° C.

In one embodiment of the second aspect of the invention, the reaction is carried out under nitrogen.

In a further embodiment of the second aspect of the invention, there is provided a process for the preparation of 8-fluoro-3-phenylsulfonylquinoline, which comprises reacting 8-fluoro-3-iodoquinoline with HSO₂Ph sodium salt in the presence of N,N′-dimethylethylenediamine, CuI, diisopropylethylamine and dimethylsulfoxide at a temperature between about 90 and about 105° C.

A third aspect of the invention provides a process for the preparation of a compound of formula (III) or a salt thereof, which comprises reacting 8-fluoroquinoline with an iodinating or brominating agent, which can act as a source of electrophilic iodine or bromine, in the presence of a solvent.

The term “iodinating agent” is intended to mean any iodine containing molecule which can act as a source of electrophilic iodine. An example of an iodinating agent is N-iodosuccinimide.

The term “brominating agent” is intended to mean any bromine containing molecule which can act as a source of electrophilic bromine. An example of a brominating agent is N-bromosuccinimide.

An example of a solvent suitable for use in a process for the preparation of a compound of formula (III) as described above is acetic acid (AcOH).

In one embodiment of the third aspect of the invention as herein described, the iodinating agent is N-iodosuccinimide and the brominating agent is N-bromosuccinimide.

In one embodiment of the third aspect of the invention as herein described, the reaction is carried out at a temperature between about 60 and about 100° C. In a further embodiment, the reaction is carried out at a temperature between about 75 and about 85° C. In yet a further embodiment, the reaction is carried out at a temperature of about 80° C.

In one embodiment of the third aspect of the invention, the reaction is carried out under nitrogen.

In one embodiment of the third aspect of the invention as herein described, once the reaction is completed, a reducing agent, for example sodium sulphite solution, is added to the reaction mix in order to reduce any remaining iodinating or brominating agent.

The term “reducing agent” is intended to mean any substance that donates electrons or a share in its electrons to another substance.

In a further embodiment of the third aspect of the invention, there is provided a process for the preparation of 8-fluoro-3-iodoquinoline, which comprises reacting 8-fluoroquinoline with N-iodosuccinimide or N-bromosuccinimide in the presence of acetic acid at a temperature between about 75 and about 85° C.

In a further embodiment of the present invention there is provided a process for the preparation of a compound of formula (I), which comprises the following steps:
(i) reacting 8-fluoroquinoline, with an iodinating or brominating agent, which can act as a source of electrophilic iodine or bromine, in the presence of a solvent to produce a compound of formula (III) and optionally adding a reducing agent once the reaction is completed;
(ii) reacting the compound of formula (III), with HSO₂Ph or a salt thereof, in the presence of a diamine ligand, a metal catalyst and a polar aprotic solvent to produce a compound of formula (II); and
(iii) reacting the compound of formula (II), with a compound of formula R¹R²NH in the presence of a base and a solvent to produce a compound of formula (I), or a salt thereof.

In one embodiment, step (iii) of the above process is carried out in the absence of a palladium catalyst, or is carried out in the absence of any metal catalyst.

In one embodiment of the present invention there is provided a process for the preparation of 3-phenylsulfonyl-8-piperazin-1-yl-quinoline, or a salt thereof, which comprises the following steps:
(i) reacting 8-fluoroquinoline with N-iodosuccinimide or N-bromosuccinimide in the presence of acetic acid at a temperature between about 75 and about 85° C. to produce 8-fluoro-3-iodoquinoline, and then adding sodium sulphite solution once the reaction is completed;
(ii) reacting the 8-fluoro-3-iodoquinoline with benzene sulfinic acid sodium salt in the presence of N,N′-dimethylethylenediamine, CuI, diisopropylethylamine and dimethylsulfoxide at a temperature between about 90 and about 105° C. to produce 8-fluoro-3-phenylsulfonylquinoline; and
(iii) reacting the 8-fluoro-3-phenylsulfonylquinoline with an excess of piperazine in the presence of potassium carbonate and n-propanol at a temperature between about 95 and about 105° C. to produce 3-phenylsulfonyl-8-piperazin-1-yl-quinoline.

This process may further include the preparation of 3-phenylsulfonyl-8-piperazin-1-yl-quinoline polymorphic Form I which comprises dissolving the 3-phenylsulfonyl-8-piperazin-1-yl-quinoline in ethyl acetate and then allowing the 3-phenylsulfonyl-8-piperazin-1-yl-quinoline to recrystallise.

The process may further include the preparation of 3-phenylsulfonyl-8-piperazin-1-yl-quinoline polymorphic Form II which comprises dissolving the 3-phenylsulfonyl-8-piperazin-1-yl-quinoline in isopropanol and then allowing the 3-phenylsulfonyl-8-piperazin-1-yl-quinoline to recrystallise.

The process may further include the preparation of 3-phenylsulfonyl-8-piperazin-1-yl-quinoline polymorphic Form III which comprises dissolving the 3-phenylsulfonyl-8-piperazin-1-yl-quinoline in ethanol and then allowing the 3-phenylsulfonyl-8-piperazin-1-yl-quinoline to recrystallise.

In any of the recrystallisation processes described above, after dissolving the 3-phenylsulfonyl-8-piperazin-1-yl-quinoline in solvent the mixture may be filtered, for example by charcoal filtration, to remove any insoluble material prior to allowing the 3-phenylsulfonyl-8-piperazin-1-yl-quinoline to recrystallise.

In any of the recrystallisation processes described above, the 3-phenylsulfonyl-8-piperazin-1-yl-quinoline solvent mixture may be seeded with 3-phenylsulfonyl-8-piperazin-1-yl-quinoline of the desired polymorphic form in order to enhance recrystallisation.

In one embodiment of the present invention there is therefore provided a process for the preparation of polymorphic Forms I, II or III of 3-phenylsulfonyl-8-piperazin-1-yl-quinoline which comprises the following steps:
(i) reacting 8-fluoroquinoline with N-iodosuccinimide or N-bromosuccinimide in the presence of acetic acid at a temperature between about 75 and about 85° C. to produce 8-fluoro-3-iodoquinoline, and then adding sodium sulphite solution once the reaction is completed;
(ii) reacting the 8-fluoro-3-iodoquinoline with benzene sulfinic acid sodium salt in the presence of N,N′-dimethylethylenediamine, CuI, diisopropylethylamine and dimethylsulfoxide at a temperature between about 90 and about 105° C. to produce 8-fluoro-3-phenylsulfonylquinoline,
(iii) reacting the 8-fluoro-3-phenylsulfonylquinoline with an excess of piperazine in the presence of potassium carbonate and n-propanol at a temperature between about 95 and about 105° C. to produce 3-phenylsulfonyl-8-piperazin-1-yl-quinoline, and
(iv) dissolving the 3-phenylsulfonyl-8-piperazin-1-yl-quinoline in:
   (a) ethyl acetate, optionally filtering the 3-phenylsulfonyl-8-piperazin-1-yl-quinoline solvent mixture, and then allowing the 3-phenylsulfonyl-8-piperazin-1-yl-quinoline to recrystallise to form Polymorphic Form I, or
   (b) isopropanol, optionally filtering the 3-phenylsulfonyl-8-piperazin-1-yl-quinoline solvent mixture, and then allowing the 3-phenylsulfonyl-8-piperazin-1-yl-quinoline to recrystallise to form Polymorphic Form II, or
   (c) ethanol, optionally filtering the 3-phenylsulfonyl-8-piperazin-1-yl-quinoline solvent mixture, and then allowing the 3-phenylsulfonyl-8-piperazin-1-yl-quinoline to recrystallise to form Polymorphic Form Ill.

In a further embodiment of the invention, the process described immediately above includes the additional step of seeding the 3-phenylsulfonyl-8-piperazin-1-yl-quinoline solvent mixture with 3-phenylsulfonyl-8-piperazin-1-yl-quinoline Polymorphic Form I in step (a), II in step (b) or III in step (c).

“Polymorphic Form I” of 3-phenylsulfonyl-8-piperazin-1-yl-quinoline is characterised in that it provides:
(i) an infrared spectrum containing peaks at 2945, 2819, 1606, 1590, 1566, 1487, 1469, 1447, 1380, 1323, 1283, 1247, 1164, 1138, 1126, 1107, 1095, 1083, 1056, 1026, 997, 964, 949, 919, 906, 879, 859, 824, 785, 761, 723, 705 cm⁻¹; and/or
(ii) a Raman spectrum containing peaks at 215, 252, 293, 304, 315, 338, 556, 705, 858, 997, 1025, 1098, 1154, 1363, 1382, 1397, 1566, 1584, 1606 and 3059 cm⁻¹; and/or
(iii) characteristic 2θ XRPD angles of 6.84, 8.61, 10.47, 13.01, 15.11, 15.90, 16.24, 16.63, 17.20, 18.00, 19.65, 21.07, 21.66, 22.20, 22.62, 23.99, 25.61, 26.12, 26.76, 27.96, 28.86, 29.64, 30.26, 30.85, 31.31, 32.60, 33.08, 33.70, 34.35, 35.65, 36.85, 38.05 and 38.46°; and/or
(iv) a melting point of 158° C.

The 2θ XRPD angles at 6.84, 8.61, 10.47, 13.01, 15.11, 15.90, 16.24, 16.63, 17.20, 18.00, 19.65, 21.07, 21.66, 22.20, 22.62, 23.99, 25.61, 26.12, 26.76, 27.96° are especially characteristic of Form I.

“Polymorphic Form II” of 3-phenylsulfonyl-8-piperazin-1-yl-quinoline is characterised in that it provides:
(i) an infrared spectrum containing peaks at 3335, 2939, 2812, 1585, 1564, 1485, 1470, 1443, 1382, 1361, 1322, 1310, 1250, 1232, 1179, 1158, 1129, 1107, 1093, 1061, 1022, 1000, 950, 914, 862, 813, 774, 760, 727 cm⁻¹; and/or
(ii) a Raman spectrum containing peaks at 216, 252, 288, 617, 701, 726, 863, 1000, 1026, 1078, 1153, 1197, 1339, 1360, 1381, 1396, 1445, 1564, 1584, and 3052 cm⁻¹; and/or
(iii) characteristic 2θ XRPD angles of 9.30, 9.95, 10.99, 13.40, 14.63, 15.03, 16.04, 16.47, 17.93, 18.19, 18.73, 19.17, 20.69, 21.49, 22.12, 23.55, 24.59, 25.27, 27.03, 28.22, 28.61, 29.48, 29.81, 30.70, 32.05, 33.32, 33.95, 34.39, 34.90, 35.77, 36.25, 36.80, 37.60, 38.19, 38.70 and 39.26°; and/or
(v) a melting point of 164° C.

The 2θ XRPD angles at 9.30, 9.95, 10.99, 13.40, 14.63, 15.03, 16.04, 16.47, 17.93, 18.19, 18.73, 19.17, 20.69, 21.49, 22.12, 23.55, 24.59, 25.27, 27.03° are especially characteristic of Form II.

“Polymorphic Form III” of 3-phenylsulfonyl-8-piperazin-1-yl-quinoline is characterised in that it provides:
(i) an infrared spectrum containing peaks at 724, 758, 777, 804, 818, 838, 856, 905, 918, 948, 1023, 1055, 1081, 1092, 1118, 1136, 1153, 1178, 1244, 1302, 1318, 1365, 1378, 1403, 1444, 1471, 1490, 1569, 1584, 1603 and 2819 cm⁻¹; and/or
(ii) a Raman spectrum containing peaks at 159, 184, 214, 241, 285, 304, 318, 429, 545, 558, 614, 706, 724, 803, 856, 1000, 1023, 1080, 1093, 1136, 1152, 1233, 1243, 1317, 1343, 1364, 1378, 1403, 1446, 1569, 1584, 1602, 3050 and 3073 cm⁻¹; and/or
(iii) characteristic 2θ XRPD angles of 10.29, 10.76, 11.94, 14.33, 14.61, 14.93, 16.02, 16.80, 17.47, 17.92, 19.13, 19.55, 19.84, 20.33, 21.16, 21.36, 23.33, 23.96, 24.44, 24.67, 25.51, 26.12, 27.13, 27.77, 28.06, 28.35, 29.23, 29.46, 30.06, 30.35, 31.27, 32.35, 32.66, 33.08, 33.77, 34.49, 35.18, 36.42, 37.34, 38.39 and 39.51°; and/or
(vi) a melting point of 188° C.

The 2θ XRPD angles at 10.29, 11.94, 17.47, 19.55, 19.84, and 20.33° are especially characteristic of Form Ill.

In a further aspect of the invention there are provided compounds of formula (II) and (III).

In one embodiment of this further aspect of the invention, there are provided the compounds 8-fluoro-3-phenylsulfonylquinoline and 8-fluoro-3-iodoquinoline. These compounds are intermediates in the processes described herein.

The compounds of formulas (I), (II) and (III), for example 3-phenylsulfonyl-8-piperazin-1-yl-quinoline, 8-fluoro-3-phenylsulfonylquinoline and 8-fluoro-3-iodoquinoline, can form acid addition salts thereof. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. The present invention includes within its scope all possible stoichiometric and non-stoichiometric forms of compounds of formulas (II) and (III), for example 8-fluoro-3-phenylsulfonylquinoline and 8-fluoro-3-iodoquinoline.

### DETAILED DESCRIPTION OF THE INVENTION

The following non-limiting Examples illustrate the process of the present invention.

### Example 1a

### Preparation of 8-fluoro-3-iodoquinoline

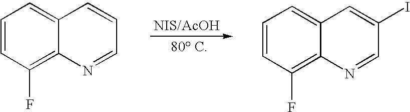

N-Iodosuccinamide (68.56 g, 305.81 mmol, 1.5 eq) was added to a solution of 8-fluoroquinoline (30 g, 203.87 mmol) in AcOH (129 ml, 4.3 vol). The mixture was stirred and heated to 80° C., under N₂ in a 250 mL CLR (Controlled Laboratory Reactor). After 24 hrs Na₂SO₃ (15 g, 0.5 weight) was added to the flask with H₂O (63 ml, 2.1 vol) and the solution was stirred, whilst be maintained at 80° C. for 1 hour to quench the remaining iodine. After an hour the reaction was allowed to cool from 80° C. to 22° C. over 30 minutes. Once 22° C. had been reached the crystals were filtered off under vacuum and washed with 2:1 AcOH/H₂O (60 ml, 2 vol) and H₂O (180 mL, 3×2 vol) and the crystals were pulled dry. The crystals were dried in an oven which was connected to an oil bath at 50° C. under reduced pressure.

The cake was removed from the oven to afford the title compound as a pale brown solid (38.63 g, 66%).

### Example 1b

### Preparation of 8-fluoro-3-iodoquinoline

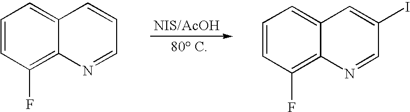

N-Iodosuccinimide (NIS) (229.0 g, 1.018 mol, 2.29 wt, 1.50 eq) was added to a stirred solution of 8-fluoroquinoline (100.0 g, 0.68 mol, 1.00 wt, 1.00 eq) in glacial acetic acid (AcOH) (430 ml, 4.3 vol). 8-Fluoroquinoline may be obtained from Orgasynth (www.orgasynth.com). The mixture was heated to circa 80° C. under nitrogen. After 23.5 hr sodium sulphite (50.0 g, 0.397 mol, 0.50 wt, 0.584 eq) and water (210 ml, 2.1 vol) were added and the mixture reheated to circa 80° C. After 1.5 hr the mixture was allowed to cool to circa 60-65° C. and seeded with 8-fluoro-3-iodoquinoline (100 mg, 0.1% wt). The product soon crystallised and the stirred slurry was allowed to cool over 1.5 hr to ambient temperature. After 1.25 hr the product was collected by vacuum filtration. The bed was washed with 1:1 acetic acid/water (2×300 ml, 3 vol) and water (2×300 ml, 2×3 vol). The bed was pulled dry for 5 min and the material used without further processing.

A sample of the material was dried in vacuo at 40-45° C., to afford the desired product in 75% yield.

¹H NMR, D₄ MeOH, 400 MHz

7.50 ppm (1H, ddd, J 1.5, 7.5 & 11.0 Hz), 7.58 ppm (1H, dt, J 5 & 8 Hz), 7.64 ppm (1H, dd, J 1.0 & 8.5 Hz), 8.78 ppm (1H, t, J 1.5 Hz), 8.99 ppm (1H, d, J 2.0 Hz)

### Example 2a

### Preparation of 8-fluoro-3-phenylsulfonylquinoline

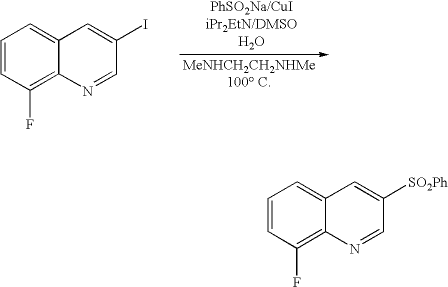

A mixture of dimethylsulfoxide (500 ml, 5 vol), 85% N,N′-dimethylethylenediamine (9.2 mL, 0.092 vol, 0.20 eq) and copper iodide (CuI) (7 g, 0.07 wt, 0.10 eq) was stirred at ambient temperature for 15 min to effect solution. Water (200 ml, 2 vol) was added and the mixture cooled to 22° C. Diisopropylethylamine (64 mL, 0.64 vol, 1.00 eq), benzenesulfinic acid sodium salt (120.0 g, 1.20 wt, 2.00 eq) and 8-fluoro-3-iodoquinoline (123.4 g of material containing 1.4% w/w AcOH and 22% w/w H₂O [equivalent to 100 g 8-fluoro-3-iodoquinoline, 1.00 wt, 1.00 eq]) were added sequentially and the resulting slurry heated under nitrogen to 100° C. over 1 hour, then maintained at 98-102° C. for 10 hr, cooled to 22° C. over 1 hour then the contents were allowed to stir for a further 1 hour. The product was collected by vacuum filtration and the cake was washed with 5:2 v/v dimethylsulfoxide—water (2×100 ml, 2×2.00 vol) and water (2×200 ml, 2×2.00 vol). The bed was pulled dry and the product dried in vacuo at 45-50° C., to give the title compound, 78.6 g, 75% yield.

¹H NMR, CDCl₃, 400 MHz

7.54-7.67 ppm, (5H, m), 7.79 ppm (1H, d, 8.0 Hz), 8.04 ppm (2H, d, 7.5 Hz), 8.86 ppm (1H, s), 9.32 ppm (1H, d, 2.0 Hz).

### Example 2b

### Preparation of 8-fluoro-3-phenylsulfonylquinoline

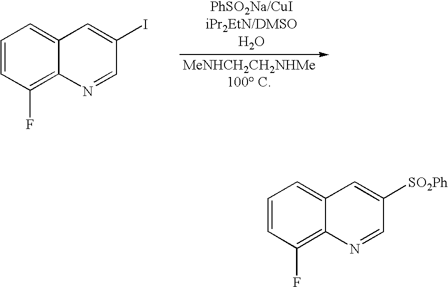

Copper iodide (CuI) (0.7 g, 0.07 wt, 0.10 eq) was added to a stirred solution of dimethylsulfoxide (50 ml, 5 vol) and 85% N,N′-dimethylethylenediamine (0.92 ml, 0.092 vol, 0.20 eq). The mixture was stirred at ambient temperature for 5 min to effect solution. Water (20 ml, 2 vol) was added (exothermic, contents increased to 40° C.) and contents maintained at 40-50° C. Diisopropylethylamine (6.4 ml, 0.64 vol, 1.00 eq), benzenesulfinic acid sodium salt (12.0 g, 1.20 wt, 2.00 eq) and 8-fluoro-3-iodoquinoline (10.0 g, 1.00 wt, 1.00 eq) were added sequentially and the resulting slurry heated under nitrogen to 100° C., then maintained at 100° C. for 12 hr. After which time the reaction mixture was cooled to 20° C. over 1 hour then aged for 5 hr at 20° C. The product was collected by vacuum filtration and the cake was washed with 5:2 v/v dimethylsulfoxide—water (2×10 ml, 2×1.00 vol) and water (2×20 ml, 2×2.00 vol). The bed was pulled dry and the product dried in vacuo at 50° C., to give the title compound, 8.04 g, 76% yield.

¹H NMR, CDCl₃, 400 MHz

7.54-7.67 ppm, (5H, m), 7.79 ppm (1H, d, 8.0 Hz), 8.04 ppm (2H, d, 7.5 Hz), 8.86 ppm (1H, s), 9.32 ppm (1H, d, 2.0 Hz).

### Example 3a

### Preparation of 3-phenylsulfonyl-8-piperazin-1-yl-quinoline

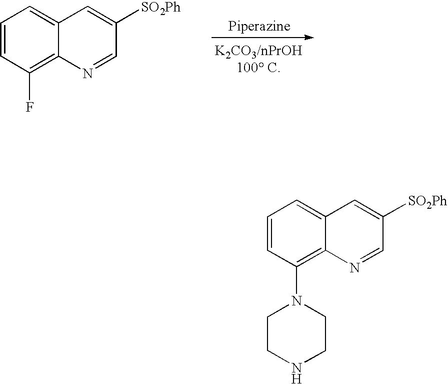

A flask was charged with 8-fluoro-3-phenylsulfonylquinoline (50.00 g, 174.03 mmol, 1.00 wt, 1.00 eq), piperazine (74.95 g, 870.14 mmol, 1.50 wt, 5.00 eq), potassium carbonate (24.05 g, 174.03 mmol, 0.48 wt, 1.00 eq) and n-propanol (100 ml, 2 vol). The mixture was stirred and heated under nitrogen at circa 100° C. After 17.25 h water (400 ml, 8 vol) was added over 1.25 h at 93-98° C. The slurry was allowed to cool to ambient temperature. After 1.5 h the product was collected by vacuum filtration. The bed was washed with 4:1 water/n-propanol (2×100 ml, 2×2 vol) and water (2×100 ml, 2×2 vol). The bed was briefly pulled dry and then the product was dried in vacuo at 50-55° C. to give the title compound, 50.92 g, 82.8% yield.

¹H NMR, CDCl₃, 400 MHz 3.17 ppm (4H, t, J 4.5 Hz), 3.34 ppm (4H, t, J 4.5 Hz), 7.27 ppm (1H, dd, J 2.0 & 7.0 Hz), 7.49-7.60 ppm (5H, m), 8.00-8.02 ppm (2H, m), 8.76 ppm (1H, d, J 2.5 Hz), 9.22 ppm (1H, d, J 2.5 Hz).

### Example 3b

### Preparation of 3-phenylsulfonyl-8-piperazin-1-yl-quinoline Form III

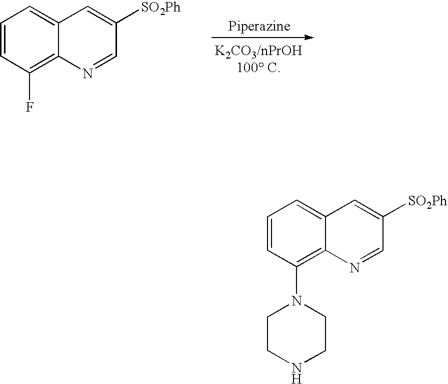

A vessel was charged with 8-fluoro-3-phenylsulfonylquinoline (20.0 g, 1.00 wt, 1.00 eq), piperazine (30.0 g, 1.50 wt, 5.00 eq), potassium carbonate (9.60 g, 0.48 wt, 1.00 eq) and n-propanol (40 ml, 2 vol). The mixture was stirred and heated under nitrogen at 100° C. After 23 h the reaction mixture was cooled to 95° C. and seeded with Form III 3-phenylsulfonyl-8-piperazin-1-yl-quinoline (20 mg, 0.001 wt, 0.001 eq) slurried in n-propanol (2×0.1 ml, 2×0.005 vol). (See WO 05/040124 for a process for making Form III 3-phenylsulfonyl-8-piperazin-1-yl-quinoline). The reaction mixture was aged at 95° C. for 15 min then cooled to 30° C. over 1 hr. Water (160 ml, 8 vol) was added over 1 hr maintaining contents at 30-34° C. The slurry was aged at 30° C. for 16 hrs then the product was collected by vacuum filtration. The bed was washed with 4:1 water/n-propanol (2×40 ml, 2×2 vol) and pulled dry. The product was dried in vacuo at 50° C. to give the title compound, 21.25 g, 86% yield.

¹H NMR, CDCl₃, 400 MHz

3.17 ppm (4H, t, J 4.5 Hz), 3.34 ppm (4H, t, J 4.5 Hz), 7.27 ppm (1H, dd, J 2.0 & 7.0 Hz), 7.49-7.60 ppm (5H, m), 8.00-8.02 ppm (2H, m), 8.76 ppm (1H, d, J 2.5 Hz), 9.22 ppm (1H, d, J 2.5 Hz).

### Example 4a

### Recrystallisation of 3-phenylsulfonyl-8-piperazin-1-yl-quinoline into polymorphic Form II

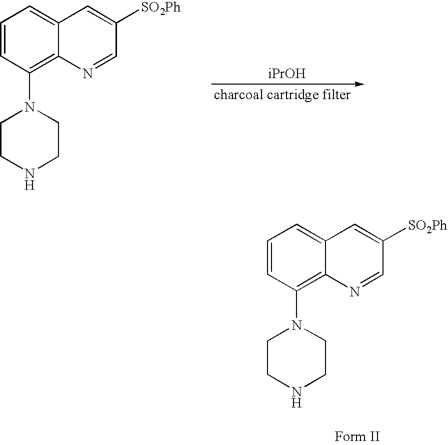

A mixture of 3-phenylsulfonyl-8-piperazin-1-yl-quinoline (80.0 g) and isopropanol (1440 mL, 18 vol) was heated to reflux for 15 min and the mixture filtered under vacuum through GF/B filter paper to remove insoluble material. The filter and flask were washed with hot isopropanol (160 mL, 2 vol), and additional isopropanol (140 mL) was added to the solution to compensate for evaporation losses in the filtration process. The filtrate was heated to reflux, resulting in the dissolution of solid which had crystallised upon cooling, then passed through a CUNO™ immobilised charcoal filter (www.cuno.com). The filter was then rinsed with refluxing isopropanol (400 mL, 5 vol). The filtrate was heated to reflux to dissolve solid which has crystallised upon cooling. The resulting solution was then cooled to 50° C. and seeded with 3-phenylsulfonyl-8-piperazin-1-yl-quinoline (Form II, 80 mg, 0.001 wt, 0.001 eq). (See WO 03/080580 for a process for making Form II 3-phenylsulfonyl-8-piperazin-1-yl-quinoline). The contents were aged for 15 min, cooled to 22° C. over 1 hr then aged at 22° C. for a further 1 hr 20 min. The contents were filtered and cake washed with isopropanol (2×80 mL, 2×1 vol). The cake was pulled dry then dried at 50° C. under reduced pressure over night to yield 3-phenylsulfonyl-8-piperazin-1-yl-quinoline, Form II, (57.9 g, 72%).

### Example 4b

### Recrystallisation of 3-phenylsulfonyl-8-piperazin-1-yl-quinoline into polymorphic Form II

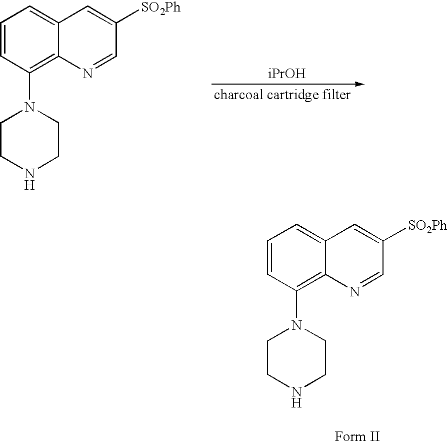

A mixture of 3-phenylsulfonyl-8-piperazin-1-yl-quinoline (813 g) and isopropanol (16.3 L, 20 vol) was heated at 80-82° C. for 35 min then passed through a CUNO™ immobilised charcoal filter (www.cuno.com), the filter was then rinsed with refluxing isopropanol (2.4 L, 3 vol). The filtrate was heated to reflux to dissolve solid which has crystallised upon cooling. The resulting solution was cooled to 63° C. and seeded with 3-phenylsulfonyl-8-piperazin-1-yl-quinoline, Form II (0.81 g, 0.001 wt, 0.001 eq) slurried in isopropanol (2×8 mL, 2×0.01 vol). (See WO 03/080580 for a process for making Form II 3-phenylsulfonyl-8-piperazin-1-yl-quinoline). The contents were aged at 63-61° C. for 15 min, cooled to 22° C. over 3 hr 45 min then aged at 22-21° C. for a further 30 min. The contents were filtered and cake washed with isopropanol (2×1.2 L, 2×1.5 vol). The cake was pulled dry then dried at 50° C. under reduced pressure to yield 3-phenylsulfonyl-8-piperazin-1-yl-quinoline, Form II, (622 g, 77%).

### Example 5a

### Recrystallisation of 3-phenylsulfonyl-8-piperazin-1-yl-quinoline into polymorphic Form II

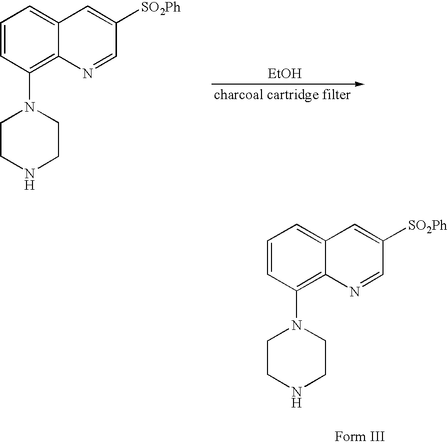

A flask was charged with 3-phenylsulfonyl-8-piperazin-1-yl-quinoline (8.4 g, 23.77 mmol) and ethanol (168 ml, 20 vol). The mixture was heated to reflux in an oil bath which was at 100° C. The solution was filtered under vacuum to remove any insoluble material and then reheated to reflux. A CUNO™ apparatus (immobilised charcoal filter) was preheated by passing through refluxing ethanol (42 ml, 5 vol). The refluxing mixture was pumped through the CUNO™ apparatus and after completion further refluxing ethanol (42 ml, 5 vol) was pumped through. Distillation was carried out on the yellow solution to reduce the volume of ethanol down to 5 vol. Once 5 vol was reached heating was stopped and the solution allowed to cool to 50° C. whilst remaining in the cooling oil bath. At 50° C. the reaction was seeded with 3-phenylsulfonyl-8-piperazin-1-yl-quinoline, Form III and left for the crystals to form with constant stirring. (See WO 05/040124 for a process for making Form III 3-phenylsulfonyl-8-piperazin-1-yl-quinoline). The solid was filtered off under vacuum and washed with ethanol (33.6 ml, 2×2 vol) and pulled dry. The cake was placed in the oven to dry at 50° C. under reduced pressure. The cake was removed from the oven to yield 3-phenylsulfonyl-8-piperazin-1-yl-quinoline (3.869 g, 46%) as a bright yellow solid.

### Example 5b

### Recrystallisation of 3-phenylsulfonyl-8-piperazin-1-yl-quinoline into polymorphic Form III

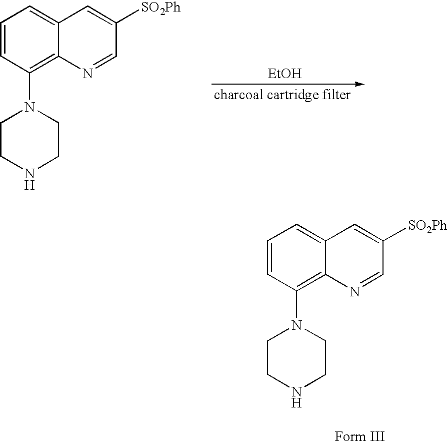

A vessel was charged with 3-Phenylsulfonyl-8-piperazin-1-yl-quinoline (1.023 Kg, 1 eq, 1 wt) and ethanol (10.2 L, 10 vol), the mixture was heated to 75° C. to dissolve the solid, then the solution was transferred to a second vessel via a 5 micron line filter. The first vessel was charged with ethanol which was heated to 72° C., the solution was transferred to the second vessel via the 5 micron line filter. The filtrate was cooled to 55° C. then seeded with 3-phenylsulfonyl-8-piperazin-1-yl-quinoline, Form III (1.0 g, 0.001 wt, 0.001 eq), the mixture was cooled to 35° C. over 45 min, held at 35° C. for 1 hr then cooled to 20° C. over 30 min. (See WO 05/040124 for a process for making Form III 3-phenylsulfonyl-8-piperazin-1-yl-quinoline). The mixture was aged at 20° C. for 1 hr 25 min then isolated via vacuum filtration. The cake was washed with ethanol (2.05 L, 2 vol) which had been cooled to 0° C., pulled dry and dried in a 50° C. oven under reduced pressure to yield 3-phenylsulfonyl-8-piperazin-1-yl-quinoline, Form III (752 g, 74%).

### Hardware for Acquiring Characterising Data of Polymorphic Forms

The infrared spectrum of polymorphic forms of 3-phenylsulfonyl-8-piperazin-1-yl-quinoline were recorded using a Nicolet Avatar 360 FT-IR spectrometer fitted with a universal Attenuated Total Reflection (ATR) accessory.

Fourier Transform (FT)-Raman spectra of polymorphic forms of 3-phenylsulfonyl-8-piperazin-1-yl-quinoline in glass tubes were acquired using a ThermNicolet 960 Enhanced Synchronization Protocol (E.S.P.) spectrometer. Excitation at 1064 nm was provided by a Nd:YVO4 laser with a power of 400 mW at the sample position. 1200 scans were recorded at 4 cm-1 resolution.

The X-Ray Powder Diffractogram pattern of the solid polymorphic forms of 3-phenylsulfonyl-8-piperazin-1-yl-quinoline was recorded using the following acquisition conditions: Unground material was packed into top-filled Si cups. Powder patterns were obtained using a Bruker D8 Advance X-Ray powder diffractometer configured with a Cu anode (40 kV, 40 mA), variable divergence slit, primary and secondary Soller slits, and a position sensitive detector. Data were acquired over the range 2-40 degrees 2-theta using a step size of 0.0145 degrees 2-theta (1 s per step). Samples were rotated during data collection.

1-13. (canceled)

14. A process for the preparation of a compound of formula (I): 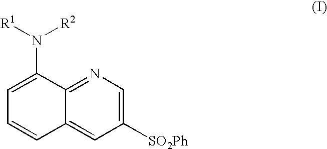
or a salt thereof, which comprises reacting a compound of formula (II): 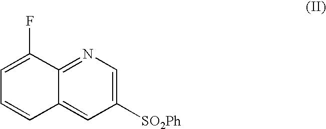
with a compound having the formula R¹R²NH, in the presence of a base and a solvent; wherein:
R¹ and R² independently represent hydrogen or C₁₋₆ alkyl, or R¹ and R² together with the nitrogen atom to which they are attached form an optionally substituted 4 to 7 membered monocyclic heterocyclyl group which can optionally contain 1 or 2 further heteroatoms selected from O, N and S; and
Ph represents an optionally substituted phenyl group.

15. A process according to claim 14, wherein the compound of formula (I) is 3-phenylsulfonyl-8-piperazin-1-yl-quinoline, and wherein the process comprises reacting 8-fluoro-3-phenylsulfonylquinoline with an excess of piperazine in the presence of potassium carbonate and n-propanol at a temperature between about 95° C. and about 105° C.

16. A process according to claim 14, further comprising reacting a compound of formula (III): 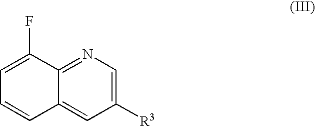
wherein R³ represents iodine or bromine; with HSO₂Ph, or a salt thereof, in the presence of a diamine ligand, a metal catalyst, a base and a polar aprotic solvent.

17. A process according to claim 16, wherein the compound of formula (II) is 8-fluoro-3-phenylsulfonylquinoline, and wherein the process comprises reacting 8-fluoro-3-iodoquinoline with HSO₂Ph sodium salt in the presence of N,N′-dimethylethylenediamine, CuI, diisopropylethylamine and dimethylsulfoxide at a temperature between about 90° C. and about 105° C.

18. A process according to claim 16, which further comprises reacting 8-fluoroquinoline, with an iodinating or brominating agent, which can act as a source of electrophilic iodine or bromine, in the presence of a solvent.

19. A process according to claim 18, which comprises reacting 8-fluoroquinoline with N-iodosuccinimide or N-bromosuccinimide in the presence of acetic acid at a temperature between about 75° C. and about 85° C.

20. A process according to claim 14, which comprises:
(i) reacting 8-fluoroquinoline with an iodinating or brominating agent, which can act as a source of electrophilic iodine or bromine, in the presence of a solvent to produce a compound of formula (III) and optionally adding a reducing agent once the reaction is completed;
(ii) reacting the compound of formula (III) with HSO₂Ph or a salt thereof, in the presence of a diamine ligand, a metal catalyst and a polar aprotic solvent to produce a compound of formula (II); and
(iii) reacting the compound of formula (II) with a compound of formula R¹R²NH in the presence of a base and a solvent to produce the compound of formula (I), or a salt thereof.

21. A process according to claim 20, wherein the compound of formula (I) is 3-phenylsulfonyl-8-piperazin-1-yl-quinoline, said process comprising:
(i) reacting 8-fluoroquinoline with N-iodosuccinimide or N-bromosuccinimide in the presence of acetic acid at a temperature between about 75° C. and about 85° C. to produce 8-fluoro-3-iodoquinoline, and then adding sodium sulphite solution once the reaction is completed;
(ii) reacting 8-fluoro-3-iodoquinoline with benzene sulfinic acid sodium salt in the presence of N,N′-dimethylethylenediamine, CuI, diisopropylethylamine and dimethylsulfoxide at a temperature between about 90° C. and about 105° C. to produce 8-fluoro-3-phenylsulfonylquinoline; and
(i) reacting 8-fluoro-3-phenylsulfonylquinoline with an excess of piperazine in the presence of potassium carbonate and n-propanol at a temperature between about 95° C. and about 105° C. to produce 3-phenylsulfonyl-8-piperazin-1-yl-quinoline.

22. A process according to claim 21, further comprising the step of dissolving 3-phenylsulfonyl-8-piperazin-1-yl-quinoline in ethyl acetate, optionally filtering the 3-phenylsulfonyl-8-piperazin-1-yl-quinoline solvent mixture, and then allowing the 3-phenylsulfonyl-8-piperazin-1-yl-quinoline to recrystallize.

23. A process according to claim 21, further comprising the step of dissolving 3-phenylsulfonyl-8-piperazin-1-yl-quinoline in isopropanol, optionally filtering the 3-phenylsulfonyl-8-piperazin-1-yl-quinoline solvent mixture, and then allowing the 3-phenylsulfonyl-8-piperazin-1-yl-quinoline to recrystallize.

24. A process according to claim 21, further comprising the step of dissolving 3-phenylsulfonyl-8-piperazin-1-yl-quinoline ethanol, optionally filtering the 3-phenylsulfonyl-8-piperazin-1-yl-quinoline solvent mixture, and then allowing the 3-phenylsulfonyl-8-piperazin-1-yl-quinoline to recrystallize.

25. A compound of formula (II) 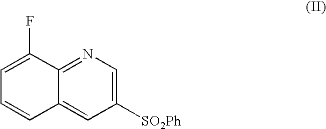
wherein Ph represents an optionally substituted phenyl group or a salt thereof.

26. A compound according to claim 25, which is 8-fluoro-3-phenylsulfonylquinoline, or a salt thereof.

27. A compound of formula (III) 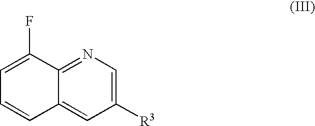
wherein R³ represents iodine or bromine or a salt thereof.

28. A compound according to claim 27, which is 8-fluoro-3-iodoquinoline, or a salt thereof.
